(19) United States
(12) Patent Application Publication
(10) Pub. No.: US 2025/0319028 A1
(43) Pub. Date: Oct. 16, 2025

(54) PHARMACEUTICAL FORMULATION COMPRISING TACROLIMUS, METHOD FOR THE PREPARATION THEREOF AND USE

(71) Applicant: PHARMATHEN S.A., Pallini Attikis (GR)

(72) Inventors: Evangelos KARAVAS, Pallini Attikis (GR); Efthymios KOUTRIS, Pallini Attikis (GR); Lida KALANTZI, Pallini Attikis (GR); Sotiria CHAITIDOU, Pallini Attikis (GR); Nikos LEMONAKIS, Pallini Attikis (GR); Anna PAPADAKI, Pallini Attikis (GR); Vincent BRIEUDES, Pallini Attikis (GR); Artemis KALEZI, Pallini Attikis (GR); Athanasios KATSENIS, Pallini Attikis (GR); Katerina KOTTI, Pallini Attikis (GR)

(73) Assignee: PHARMATHEN S.A., Pallini Attikis (GR)

(21) Appl. No.: 18/695,608

(22) PCT Filed: Sep. 27, 2022
(86) PCT No.: PCT/EP2022/025445

§ 371 Date: Mar. 26, 2024

(30) Foreign Application Priority Data
Sep. 27, 2021 (GR) 20210100639
Nov. 10, 2021 (GB) 2116138.5

(51) Int. Cl. A61K 9/16; A61K 31/436

(52) U.S. Cl. CPC A61K 9/1647; A61K 9/1694; A61K 31/436

(57) ABSTRACT

A long acting injectable formulation based on combination of biodegradable poly(D,L-lactide-co-glycolide) microparticles comprising different PLGA polymers and Tacrolimus. The microparticles may include Tacrolimus, a first polymer and a second polymer, and the first and second polymer may differ from each other. Each of the first and second polymers may be a poly(D,L-lactide-co-glycolide) polymer. Each of the first and second polymers may have an identical lactide to glycolide ratio. Each of the first and second polymers may have a different molecular weight

### TECHNICAL FIELD OF THE INVENTION

The present invention relates to a stable extended-release injectable pharmaceutical formulation containing a therapeutically effective quantity of Tacrolimus, a method for the preparation thereof and use of the formulation for treatment and prevention of organ rejection after transplantation, graft-versus-host diseases by medulla ossium transplantation, autoimmune diseases, infectious diseases, and the like.

### BACKGROUND OF THE INVENTION

Transplant rejection is a process in which a transplant recipient's immune system attacks the transplanted organ or tissue. The more similar the antigens are between the donor and recipient, the less likely that the organ will be rejected. Tissue typing ensures that the organ or tissue is as similar as possible to the tissues of the recipient. The match is usually not perfect. No two people, except identical twins, have identical tissue antigens.

Following transplantation medicines are used to suppress the recipient's immune system. The goal is to prevent the immune system from attacking the newly transplanted organ. If these medicines are not used, the body will almost always launch an immune response and reject the foreign tissue.

Immunosuppressants or antirejection drugs lower the body's ability to reject a transplanted organ. There are two types of immunosuppressants: induction drugs are powerful antirejection medicines used at the time of transplant and maintenance drugs are antirejection medications used typically soon after transplantation and for the long term. Commonly used maintenance drugs are calcineurin inhibitors (CNI). Tacrolimus is the CNI immunosuppressant used by the majority of transplant patients based on the reports from the Systematic Registry of Transplant Recipients (SRTR).

The immunosuppressive activity of Tacrolimus is mediated through the inhibition of calcineurin, which is a protein phosphatase found in the cytoplasm of T-cells, and the subsequent blockage of interleukin-2 production, leading to a decrease in T cell proliferation.

The molecular formula of Tacrolimus monohydrate is C₄₄H₆₉NO₁₂·H₂O, corresponding to a molecular weight of 822. It is a white or almost white crystalline powder. It is freely soluble in ethanol and practically insoluble in heptane and water.

Tacrolimus in aqueous solution epimerizes to an intermediate Tacrolimus compound-I which is converted into Tacrolimus compound-II to reach an equilibrium containing three forms. This is an inherent property of the molecule.

Tacrolimus is currently available under the brand name PROGRAF™ in oral tablets, capsules and suspension and as concentrate for solution for infusion only for hospitalized patients. The solution for infusion comprises polyoxyl 60 hydrogenated castor oil or polysorbate 80 as solubilizers the presence of which can lead to anaphylactic shock (i.e. severe allergic reaction) and death in patients. Directions for using intravenous infusions recommend that patients should be converted from intravenous to oral medication as soon as individual circumstances permit to avoid anaphylactic reactions, the intravenous therapy should not be continued for more than 7 days. Oral medications are administered at least once daily.

WO 2006/002365 A2 discloses a formulation comprising a microparticle wherein the microparticle comprises a polymer and a drug such as Tacrolimus, and wherein the drug is present in the microparticle at a concentration of greater than 50% and preferably greater than 75% (weight of drug/weight of microparticle) suggesting that high loading formulations may facilitate less frequent dosing. Unfortunately, the formulations disclosed have the disadvantages that at least 15% of the drug is released almost immediately and release of the drug lasts for only up to about 3 weeks.

EP 1868576 A2 discloses, as a way to avoid the anaphylaxis problem, a hydrogenated castor oil free injectable nanoparticulate formulation comprising: (a) particles of Tacrolimus having an effective average particle size of less than about 2000 nm; and (b) at least one surface stabilizer. Unfortunately use of the particle sizes disclosed has the disadvantage that such particles will be phagocytosed by immune cells (Dawes G. J. S. et al Mater Sci: Mater Med (2009) 20:1089-1094)

Although each of the patents above represents an attempt to overcome the problems associated with existing treatment regimens, they do not provide a suitable controlled release product and there still exists a need for controlled release injectable formulations that avoid plasma fluctuations, avoid high initial release of the drug, provide satisfactory levels of release, reduce the risks of associated side effects such as anaphylaxis, and avoid having to remember to take daily doses of oral products and thereby improve patient compliance.

### SUMMARY OF THE INVENTION

The present invention provides a pharmaceutical formulation comprising microparticles wherein the microparticles comprise two different polymers and Tacrolimus, wherein each of the polymers is a poly(D,L-lactide-co-glycolide) polymer and each of the polymers has the same lactide to glycolide ratio and each of the polymers has a different molecular weight.

Tacrolimus according to the present invention can include the base or any salt of Tacrolimus, in any crystalline or amorphous form, or a derivative thereof. Two kinds of conformational heterogeneity of Tacrolimus have been reported:
1) cis-trans conformational isomerization involving restricted rotation of the amide bond in the pipecolic moiety.
2) cis isomer and Tacrolimus exist in cis conformation in the solid state.

The present invention is directed to an injectable pharmaceutical formulation for controlled release of Tacrolimus, for parenteral administration that is used to prevent or treat organ rejection after transplantation, more particularly for the prophylaxis of organ rejection in adult and pediatric patients receiving allogeneic liver, kidney or heart transplants, optionally in combination with other immunosuppressants.

The object of the present invention is to provide Tacrolimus encapsulated into polymeric microparticles in order to control the release of the drug and reduce the administration frequency. Such a formulation ensures better medication adherence, decreases the need for therapeutic drug monitoring, reduces the possibility of anaphylaxis issues with the presently infused tacrolimus formulations and avoids the need for daily dosing of an oral product.

A further advantage of the present invention is that it provides a Tacrolimus injectable formulation that does not exhibit any release lag phase or burst and substantially has a linear release profile for a period of up to two months. This is achieved by combining two microparticle types made of different PLGA polymers.

A further object of the present invention is to provide an injectable formulation that can be administered subcutaneously or intramuscularly to form a depot that provides long term controlled release of the drug.

A further object of the present invention is to provide an injectable controlled release formulation comprising Tacrolimus, as an active ingredient, which shows good syringability, injectability, no clogging or blocking of the syringe needles, good drainage, sterility and re-suspendibility in case of suspensions.

A further object of the present invention is to provide a method of preparing injectable polymeric microparticles in powder form comprising Tacrolimus. The method comprises emulsification (o/w) (single or double) followed by solvent extraction/evaporation. An aqueous vehicle is also provided for powder reconstitution before administration.

The microparticles with the diluent can exist in a dual chamber syringe or as a kit having syringe pre-filled with the diluent and microparticles existing in a separate vial.

Other objects and advantages of the present invention will become apparent to those skilled in the art in view of the following detailed description.

### DETAILED DESCRIPTION OF THE INVENTION

For the purposes of the present invention, a pharmaceutical formulation comprising an active ingredient is considered to be stable if the active ingredient degrades less or more slowly than it does on its own and/or in known pharmaceutical formulations. The words controlled, extended, sustained and long-acting release are used interchangeably unless otherwise stated.

As already mentioned, the main object of the present invention is to provide a controlled release injectable formulation of Tacrolimus in the form of drug-loaded microparticles that contribute to pharmacokinetic optimization of Tacrolimus and improvement of medication adherence.

In spite of its success in ensuring graft survival, the therapeutic use of tacrolimus is complicated due to its narrow therapeutic index (between 5 and 15 ng/ml). Tacrolimus has a large inter-/intra-patient variability in pharmacokinetics profile and a poor oral bioavailability because of its poor solubility. Sub-therapeutic level of Tacrolimus may result in acute rejection of xenografts. Moreover, systemically delivered Tacrolimus may cause severe side effects including nephrotoxicity and global immunosuppression owing to the non-selective distribution of the drug. In fact, drug-induced nephrotoxicity is the major dose-limiting side effect of TAC with a reported overall incidence as high as 44%. Unfortunately, nephrotoxicity can lead to severe complications such as negative impact on graft survival and life expectancy of the patients. Indeed, nephrotoxic effects present challenges during therapeutic regimen with these drugs (Randhawa, P. S., Starzl, T. E. & Demetris, A. J. Tacrolimus (FK506)-Associated Renal Pathology. Adv Anat Pathol 4, 265-276 (1997)).

Tacrolimus is currently available in oral dosage forms including immediate release capsules, extended release capsules and extended release tablets. Low aqueous solubility, site dependent permeability, extensive first pass metabolism in the gut and liver, P-gp mediated drug efflux and influence of food are the most important reasons for low and variable oral bioavailability of Tacrolimus. While Tacrolimus is also available as concentrate for solution for infusion, the intravenous administration is only limited to early stages of organ transplantation when oral administration is not feasible and when the subject is still under hospital care, it is recommended (Prograf® injection USA prescribing information) that intravenous infusion should be discontinued as soon as the patient can tolerate oral administration.

The present invention provides a controlled release drug delivery system for parenteral administration of Tacrolimus in a biodegradable polymer as microparticles enabling the active ingredient's sustained release after residence time in the polymer that controls the drug release and reduces the associated toxicities while maintaining the immunosuppressive activity of Tacrolimus and avoids the poor oral bioavailability issues described above.

Adherence to treatment is an important determinant of clinical outcomes for patients in a wide range of clinical settings. Adherence is particularly important in severe illnesses in which patients often require treatment for months or years and premature discontinuation of treatment can have serious consequences for patient health and quality of life.

Regardless of the specific reason for treatment nonadherence, the failure of patients to continue to take medication as prescribed contributes to high rates of relapse, hospitalization, and in some patients an increased risk of death.

Recent advances in drug delivery technologies have led to the development of innovative delivery systems designed to improve therapeutic outcomes. One possible solution to the problem of poor adherence to pharmacotherapy is the development of new, long-acting drug-delivery systems, which gradually release medication over a period of several days or weeks with a single application. Long-acting injectable technologies may offer superiority over conventional products by improving safety and efficacy through prolonged duration of action and reducing adherence issues as well as side effects. By enabling patients to take medications less frequently, these technologies create drugs that can be especially beneficial in treating severe diseases in which medication compliance is closely correlated with improved outcomes.

The formulations of the present invention have enhanced solubility characteristics, which, in turn, provide enhanced bioavailability upon administration to a patient, as well as reduced absorption variability. By satisfying these needs the present invention eliminates the need to use polyoxyl 60 hydrogenated castor oil (HCO-60) and/or polysorbate 80 as solubilizers. This is beneficial, as conventional injectable Tacrolimus formulations comprise polyoxyl 60 hydrogenated castor oil or polysorbate 80 as solubilizers. The presence of such solubilizing agents can lead to anaphylactic shock (i.e. severe allergic reaction) and death in patients.

The present invention is used for the prophylaxis of transplant rejection in adult kidney, liver or heart allograft recipients. A therapeutically effective amount of the injectable formulation of the present invention is administered to the subject so as to form a subcutaneous or intra-muscular depot within the patient. The depot slowly releases Tacrolimus over time to provide long treatment to the allogenic organ recipient.

Biodegradable materials are natural or synthetic in origin and are degraded in vivo, either enzymatically or non-enzymatically or both, to produce biocompatible, toxicologically safe by-products which are further eliminated by the normal metabolic pathways. The number of such materials that are used in controlled drug delivery has increased dramatically over the past decade. The basic category of biomaterials used in drug delivery can be broadly classified as (1) synthetic biodegradable polymers, which includes relatively hydrophobic materials such as the α-hydroxy acids (a family that includes poly lactic-co-glycolic acid, PLGA), polyanhydrides, and others, and (2) naturally occurring polymers, such as complex sugars (hyaluronan, chitosan) and inorganics (hydroxyapatite).

Polyester PLGA is a copolymer of poly lactic acid (PLA) and poly glycolic acid (PGA). It is the best-defined biomaterial available for drug delivery with respect to design and performance. Poly lactic acid contains an asymmetric α-carbon which is typically described as the D or L form in classical stereochemical terms and sometimes as R and S form, respectively. The enantiomeric forms of the polymer PLA are poly D-lactic acid (PDLA) and poly L-lactic acid (PLLA). PLGA is generally an acronym for poly D,L-lactic-co-glycolic acid where D- and L-lactic acid forms are in equal ratio.

Injectable biodegradable and biocompatible PLGA particles (microparticles, microcapsules, nanocapsules, nanospheres) may be employed for controlled-release dosage forms. Drugs formulated in such polymeric devices are released either by diffusion through the polymer barrier, or by erosion of the polymer material, or by a combination of both diffusion and erosion mechanisms. In addition to its biocompatibility, drug compatibility, suitable biodegradation kinetics and mechanical properties, PLGA can be easily processed and fabricated in various forms and sizes. Polymer formulation is the most important factor to determine the hydrophilicity and rate of degradation of a delivery matrix which influence the rate of degradation. An increase in glycolic acid percentage in the oligomers generally accelerates the weight loss of polymer. PLGA 50:50 exhibits a faster degradation than PLGA 65:35 due to preferential degradation of glycolic acid proportion assigned by higher hydrophilicity. Subsequently PLGA 65:35 shows faster degradation than PLGA 75:25 and PLGA 75:25 than PLGA 85:15. Thus the absolute value of the degradation rate increases with the glycolic acid proportion. The amount of glycolic acid is a critical parameter in tuning the hydrophilicity of the matrix and thus the degradation and drug release rate. Polymers with higher molecular weight generally exhibit lower degradation rates. Molecular weight has a direct relation with the polymer chain size. Polymers having higher molecular weight have longer polymer chains, which require more time to degrade than small polymer chains.

As drug release rate and release time can be regulated by adjusting polymer type, polymer molecular weight and microsphere size and morphology, it is possible to fabricate drug-loaded microparticles according to therapeutic needs. There are two anticipated effects of the application of PLGA microsphere technology to Tacrolimus. One is a reduction in adverse effects associated with a change of pharmacokinetic profile. The other is improved medication adherence.

Suitable commercially obtainable polymers for use in preparing of PLGA microparticles according to the present invention include but are not limited to RESOMER® and LAKESHORE BIOMATERIALS by Evonik Industries AG, Expansorb® by PCAS., PURASORB® by PURAC Biochem BV.

The purposes of the present invention are particularly assisted by the use of PLGA polymers having a 50:50 lactide to glycolide ratio. Such polymers, preferably those having molecular weight from 15,000 to 80,000 Da, more preferably from 15,000 to 58,000 Da, and especially those of molecular weight of approximately from 17,000 Da to 50,000 Da are of particular relevance in achieving the linear release profile for at least a two months period.

In a preferred embodiment of the present invention the molecular weight of the first polymer is from 15,000 to 30,000 Da, and the molecular weight of the second polymer is from 30,000 to 80,000 Da. In a further preferred embodiment of the present invention the molecular weights of the two polymers are 17,000 Da and 50,000 Da respectively.

The use of a single PLGA polymer could not give the desired release profile but now surprisingly we have found that a linear profile of Tacrolimus release controlled to give a low initial burst release of Tacrolimus and controlled for a period of at least two months is achieved when two different PLGA microparticle types are combined. The PLGA polymer in both types has a 50:50 lactide to glycolide ratio however each microparticle type is prepared with polymer of different molecular weight. When the two microparticle types are combined in ratios of from 70:30 to 30:70 the required release rate is achieved.

Nevertheless, appropriate choice & combination of polymers remains to be seen if they could function in a similar manner. It can be proven that combination of microparticles manufactured with more than two different polymers of the same or different nature, of different molecular weight and/or lactide to glycolide ratio can present behavior comparable to that of the present invention. Furthermore, the present invention might find application to other pharmaceutically active ingredients of low solubility and high membrane permeability like Tacrolimus. Such pharmaceutically active ingredients could be flurbiprofen, naproxen, cyclosporin, ketoprofen, rifampicin, carbamazepine glibenclamide, bicalutamide, ezetimibe, aceclofenac and others.

Amount of drug loading as well as polymer concentration in the drug delivery matrix plays a significant role on the rate and duration of drug release. Matrices having higher drug content possess a larger initial burst release than those having lower content because of their smaller polymer to drug ratio. However, this drug content effect is attenuated when the drug content reaches a certain level depending upon drug type. In the present invention a drug loading in the microparticles of below 30% w/w is preferable, especially from 20% to 30% w/w of Tacrolimus. A polymer concentration from 5% to 13% w/w is also in preferred in the present invention.

A number of process for making the PLGA microparticles are known. Preferably the microparticles of the present invention are produced by a single emulsion solvent evaporation process. This is the easiest, fastest and most cost-effective process. Suitable processes are described in more detail below:
a) A process for the preparation of microparticles comprises the following steps:
   two different molecular weight PLGA polymers are dissolved under stirring in a suitable solvent
   Tacrolimus is added in the polymer solution under stirring to form a dispersed oil phase (DP);
   a continuous phase (CP) comprising water for injection (WFI), one or more surfactants and one or more buffering agents is prepared and kept under controlled temperature. The continuous phase is thermostatted at a temperature lower than 20° C., more preferably from 5 to 10° C.;
   The dispersed and the continuous phases are mixed and emulsified using a high shear rotor-stator continuous flow disperser (i.e., in line homogenizer) or an overhead stirrer to form a suspension;
   The suspension is subjected to solvent extraction and evaporation by stirring under controlled temperature and air flow to ensure satisfactory removal of organic solvents and microparticles solidification;
   The formed microparticles are collected onto sieves and washed with water;
   The microparticles are dried under vacuum.
b) A process for the preparation of microparticles comprising the following steps:
   i) —a first PLGA polymer is dissolved under stirring in a suitable solvent;
   Tacrolimus is added in the polymer solution under stirring to form a dispersed oil phase (DP);
   a continuous phase (CP) comprising water for injection (WFI), one or more surfactants and one or more buffering agents is prepared and kept under controlled temperature. The continuous phase is thermostatted at a temperature lower than 20° C., more preferably from 5 to 10° C.;
   The dispersed and the continuous phases are mixed and emulsified using a high shear rotor-stator continuous flow disperser (i.e., in line homogenizer) or an overhead stirrer to form a suspension;
   ii) a second PLGA polymer with a different molecular weight to the first polymer is dissolved under stirring in a suitable solvent
   Tacrolimus is added in the polymer solution under stirring to form a dispersed oil phase (DP);
   a continuous phase (CP) comprising water for injection (WFI), one or more surfactants and one or more buffering agents is prepared and kept under controlled temperature. The continuous phase is thermostatted at a temperature lower than 20° C., more preferably from 5 to 10° C.;
   The dispersed and the continuous phases are mixed and emulsified using a high shear rotor-stator continuous flow disperser (i.e., in line homogenizer) or an overhead stirrer to form a suspension;
   iii) the suspensions containing the first PLGA polymer and the second PLGA polymer are mixed together and subjected to solvent extraction and evaporation by stirring under controlled temperature and air flow to ensure satisfactory removal of organic solvents and microparticles solidification;
   The formed microparticles are collected onto sieves and washed with water;
   The microparticles are dried under vacuum.
c) A process for the preparation of microparticles of claim 1 comprising the following steps:
   i) —a first PLGA polymer is dissolved under stirring in a suitable solvent;
   Tacrolimus is added in the polymer solution under stirring to form a dispersed oil phase (DP);
   a continuous phase (CP) comprising water for injection (WFI), one or more surfactants and one or more buffering agents is prepared and kept under controlled temperature. The continuous phase is thermostatted at a temperature lower than 20° C., more preferably from 5 to 10° C.;
   The dispersed and the continuous phases are mixed and emulsified using a high shear rotor-stator continuous flow disperser (i.e., in line homogenizer) or an overhead stirrer to form a suspension;
   The suspension is subjected to solvent extraction and evaporation by stirring under controlled temperature and air flow to ensure satisfactory removal of organic solvents and microparticles solidification;
   The formed microparticles are collected onto sieves and washed with water;
   The microparticles are dried under vacuum.
   ii) a second PLGA polymer with a different molecular weight to the first polymer is dissolved under stirring in a suitable solvent;
   Tacrolimus is added in the polymer solution under stirring to form a dispersed oil phase (DP);
   a continuous phase (CP) comprising water for injection (WFI), one or more surfactants and one or more buffering agents is prepared and kept under controlled temperature. The continuous phase is thermostatted at a temperature lower than 20° C., more preferably from 5 to 10° C.;
   The dispersed and the continuous phases are mixed and emulsified using a high shear rotor-stator continuous flow disperser (i.e., in line homogenizer) or an overhead stirrer to form a suspension;
   The suspension is subjected to solvent extraction and evaporation by stirring under controlled temperature and air flow to ensure satisfactory removal of organic solvents and microparticles solidification;
   The formed microparticles are collected onto sieves and washed with water;
   The microparticles are dried under vacuum.
   iii) after drying the microparticles made from the first PLGA polymer and the microparticles made from the second PLGA polymer are physically mixed.

The molar ratio of the PLGA polymer may be from 70:30 to 30:70, preferably a molar ratio of 50:50.

Suitable solvents for the PLGA that can be used in the above processes include but are not limited to organic solvents such as ethylacetate, tetrahydrofuran, acetonitrile, dichloromethane (DCM) and chloroform, a preferred solvent is dichloromethane.

The continuous phase consists of an aqueous solution with one or more surfactants, selected from anionic surfactants (such as sodium stearate, sodium lauryl sulfate), non-ionic surfactants (such as tweens), polyvinylpyrrolidone, carboxymethylcellulose sodium and gelatin, used independently or in combination. It is preferred to use one surfactant. A preferred surfactant is polyvinyl alcohol (PVA).

Suitable buffering agents include sodium carbonate, sodium bicarbonate, potassium carbonate, potassium bicarbonate, sodium hydrogen phosphate, sodium dihydrogen phosphate, potassium phosphate monobasic, and potassium phosphate dibasic and combinations thereof, preferred buffering agents are sodium carbonate and sodium bicarbonate and a combination thereof.

The process described by the present invention results in the formation of microparticles with a particle size distribution of 10-200 microns measured by laser light diffraction.

The formulations are preferably administered by subcutaneous or intramuscular injection after being reconstituted with suitable diluent. More particularly the diluent may be packed in pre-filled syringe and the powder containing the microparticles in a vial. Immediately before use the content of pre-filled syringe (solvent) and vial (powder) are mixed to prepare the suspension to be injected to the patient. Alternatively, a dual chamber pen may be used; the powder in one chamber is mixed before use with the solvent in the other chamber of the pre-filled pen and the obtained suspension is injected to the patient. The formulations are preferably administered once every two months.

Suitable diluents include pharmaceutically acceptable excipients selected from the group consisting of suspending agents/viscosity enhancers, buffering agents and/or pH-adjusting agents, surfactants, and tonicity-adjusting agents. Suitable viscosity enhancing agents mannitol, include sodium carboxymethyl cellulose, polyvinylpyrrolidone (PVP), such as PLASDONE and hydroxypropylmethylcellulose (HPMC), such as Methocel, preferably sodium carboxymethyl cellulose and mannitol. Commonly used buffer excipients include citric acid monohydrate, glycine, maleic acid, methionine, sodium acetate, sodium citrate dihydrate, sodium dihydrogen phosphate monohydrate and disodium phosphate heptahydrate preferably sodium dihydrogen phosphate monohydrate and disodium phosphate heptahydrate and/or citric acid monohydrate. Tonicity-adjusting agents such as dextrose, mannitol, potassium chloride, sodium chloride may be used preferably sodium chloride. Surfactants may also be used, for example polysorbate 20 and 80, D-a-tocopheryl polyethylene glycol 1000 succinate, polyoxyethylated castor oil preferably polysorbate 20 and 80. PH-adjusting agents are selected from acetic acid, sodium hydroxide, sodium chloride preferably sodium hydroxide and/or sodium chloride. Aqueous diluents are preferred particularly those with a pH range of 6-7.5 and viscosity in the range between 3-90 cP.

### EXAMPLES

### Example 1

The compatibility of materials was examined by dissolving the polymers in various solvents (i.e., DCM, THF) and adding slowly the API material in the produced solution. Different polymers were used as presented in Table 1. Clear solutions were obtained in all cases with a Tacrolimus concentration up to 30% w/w.

For the degradation studies, films of each polymer containing 30% w/w Tacrolimus were synthesized and compared with placebo films (without the presence of Tacrolimus). The films were prepared from a solution containing the appropriate amounts of the polymer and Tacrolimus in DCM solvent after evaporating the solvent. The isolated films were immersed in a phosphate buffer saline (PBS, pH=7.4) solution and kept at 37° C. for almost one month. At regular time intervals, samples of the films were withdrawn, and the Mw was measured with GPC to examine the mass loss.

TABLE 1
Polymers used in the compatibility and degradation studies

| | | End | Molecular |
|---|---|---|---|
| Name | L:G Ratio | Group | Weight (Da) |
| PURASORB | 50:50 | Acid | ≈17,000 |
| PDLG 5002A | | | |
| RESOMER | 50:50 | Acid | ≈50,000 |
| RG 504H | | | |
| RESOMER | 75:25 | Ester | ≈40,000 |
| RG 753S | | | |

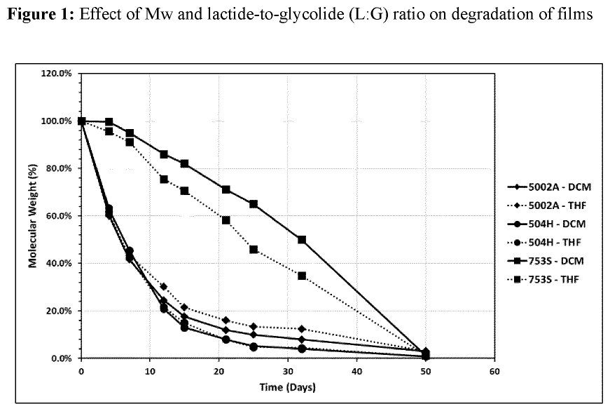

The films were homogenous, and no phase separation was observed. The polymer MW loss in terms of time was measured and the results are presented in FIG. 1.

Polymers with the same Lactide:Glycolide ratio of 50:50 are the most suitable for the purposes of the present invention. Moreover, all films were studied for their compatibility with Tacrolimus & presented very similar degradation profiles indicating that there is no API-induced degradation.

### Example 2

Microparticles of the two polymers of the same lactide to glycolide ratio were prepared using a single emulsion solvent evaporation process as follows.

Poly(D,L-lactide-co-glycolide) with a molar ratio of 50:50 (Mw=17,000 or Mw=50,000), was dissolved in dichloromethane under stirring. Tacrolimus was subsequently dissolved in the polymer solution to form the dispersed phase (DP). Poly(vinyl alcohol) was dissolved in water for injection at 80° C. followed by the addition of sodium hydrogen carbonate and sodium carbonate. The solution was cooled down to 25° C. to form the continuous phase (CP). Microparticles of the desired particle size distribution were prepared by delivering the CP and the DP into an in-line disperser. The suspension was subjected to solvent extraction and evaporation by stirring under controlled temperature at 20° C. and air flow to ensure removal of organic solvents and particles solidification. After 3 to 4 hours the microparticles were transferred to a glass filter dryer, washed with an excess of water at room temperature and left under vacuum for 24 hours to dry.

The effect of the Mw of the polymer used, on the quality attributes (i.e., particle size and release profile) of the produced microparticles, is shown on Table 2.

TABLE 2
Drug loading, PSD and release rate
of the formulated microparticles

| | Exp. | | | | | | |
|---|---|---|---|---|---|---|---|
| | Drug | Particle Size Distribution | | | % Release/ | | |
| PLGA | Loading | (μm) | | | Days | | |
| MW | (%) | *Dv(10) | Dv(50) | Dv(90) | 10% | 50% | 80% |
| 5002A | 29.37 | 49.22 | 73.68 | 108.21 | 1.80 | 16.09 | 23.46 |
| (17,000 | | | | | | | |
| Da) | | | | | | | |
| 504H | 28.43 | 77.36 | 112.00 | 158.22 | 25.00 | 34.64 | ≥50 |
| (50,000 | | | | | | | |
| Da) | | | | | | | |

*Dv(x) percentile of the cumulative volume distribution as measured by laser light diffraction.

The results reveal that higher molecular weight polymers lead to larger microparticles for the same set of process parameters. This can be attributed to the higher viscosity of the dispersed phase during emulsification. The earlier release profile obtained by smaller microparticles can be attributed to the higher surface area to unit volume ratio.

### Example 3

The effect of polymer concentration in the dispersed (oil) phase during microparticles formulation was also examined for the low MW PLGA polymer. Formulations have been tested to evaluate the effect of PLGA concentration on the quality attributes of the microparticles.

The effect of polymer concentration in the dispersed (oil phase), on the quality attributes (i.e. particle size and the release profile) of the produced microparticles is shown on Table 3.

TABLE 3
Drug loading, PSD and release rate
of the formulated microparticles

| | Exp. | | | | | | |
|---|---|---|---|---|---|---|---|
| PLGA | Drug | Particle Size Distribution | | | % Release/ | | |
| Conc. | Loading | (μm) | | | Days | | |
| (% w/w) | (%) | Dv(10) | Dv(50) | Dv(90) | 10% | 50% | 80% |
| 13 | 29.37 | 49.22 | 73.68 | 108.21 | 1.80 | 16.09 | 23.46 |
| 5 | 24.84 | 15.39 | 25.80 | 39.88 | 0.72 | 15.32 | 23.55 |

As presented in table 3 above by increasing the polymer concentration of the dispersed phase, the particle size is also increased due to the higher viscosity during emulsification. The dissolution profiles of the two formulations were almost similar with slightly higher linearity for the microparticles obtained with lower PLGA concentration. Therefore, it appears that a PLGA concentration between 5% and 13% w/w creates microparticles with an acceptable size and are appropriate for the purposes of the present invention.

### Example 4

As shown by the previous formulations, the particle size plays an important role on the dissolution profile. Apart from the particle size, the surface area per unit volume can also be increased by creating porous in the microparticles. The most common practice in order to create porous microparticles using the solvent extraction/evaporation method is to apply the double emulsion process. In order to evaluate the effect of porosity on the release rate, a double emulsion solvent extraction and evaporation process was applied, and the produced formulations were compared with the those performed using the single emulsion process.

The effect of the emulsion type on the quality attributes (i.e. particle size and the release profile) of the produced microparticles is shown on Table

TABLE 4
Drug loading, PSD and release rate
of the formulated microparticles

| | Exp. | | | | | | |
|---|---|---|---|---|---|---|---|
| | Drug | Particle Size Distribution | | | % Release/ | | |
| Emulsion | Loading | (μm) | | | Days | | |
| Type | (%) | Dv(10) | Dv(50) | Dv(90) | 10% | 50% | 80% |
| Single | 30.61 | 15.1 | 25.1 | 38.7 | 4.63 | 14.58 | 20.60 |
| Double | 29.67 | 31.4 | 43.1 | 58.6 | 3.51 | 11.51 | 17.78 |

Double emulsion process has led to larger microparticles compared to single emulsion with the same set of process parameters. From the results obtained based on size we would expect to see higher release rates for smaller particles. The results show an earlier release profile for larger microparticles obtained by the double emulsion process. This reveals that the existence of porosity prevails over the size effect and the larger porous microparticles exhibit higher release rates due to the higher specific surface area.

In order to evaluate more conclusively the effect of porosity on the release rate, microparticles exhibiting almost identical sizes should be tested. In order to isolate microparticles with the same size, in-line homogenizer was utilized.

The effect of the emulsion type on the quality attributes (i.e., particle size and the release profile) of the produced microparticles is shown on Table 5.

TABLE 5
Drug loading, PSD and release rate
of the formulated microparticles

| | Exp. | | | | | | |
|---|---|---|---|---|---|---|---|
| | Drug | Particle Size Distribution | | | % Release/ | | |
| Emulsion | Loading | (μm) | | | Days | | |
| Type | (%) | Dv(10) | Dv(50) | Dv(90) | 10% | 50% | 80% |
| Single | 27.16 | 4.54 | 10.45 | 20.78 | 1.51 | 9.24 | 16.06 |
| Double | 27.49 | 4.84 | 10.72 | 22.37 | 0.84 | 4.31 | 9.04 |

From the results presented it is obvious that for microparticles of the same size the dissolution profile is much faster for microparticles obtained by the double emulsion process. This is attributed to the clear effect of the porosity of the microparticles and their higher specific surface area due to porosity.

### Example 5

In order to achieve a linear profile for a period of 2 months according to the present invention, microparticles using the two PLGAs of different MW but same (50:50) lactide to glycolide ratio were prepared. The single emulsification process as that described in example 2 was used. The concentration of PLGA was between the optimum ratio of 5% to 13% w/w, the concentration of Tacrolimus was below 30% w/w and the microparticles created had a particle size between 10 and 200 microns.

The created separately microparticles were physically mixed at different mass ratios ranging from 70:30 to 30:70 and their dissolution profile was measured and presented in table 6 below.

TABLE 6
Release profiles of the different microparticle
physical mixtures in different mass ratios

| | | |
|---|---|---|
| Mixture | Mixture | Mixture |
| (50:50) | (70:30) | (30:70) |
| (5002A:504H) | (5002A:504H) | (5002A:504H) |

| Time | Release | Time | Release | Time | Release |
|---|---|---|---|---|---|
| (Days) | (%) | (Days) | (%) | (Days) | (%) |
| 0.0 | 0.0 | 0.0 | 0.0 | 0.0 | 0.0 |
| 0.1 | 1.3 | 0.1 | 1.3 | 0.1 | 1.2 |
| 1.0 | 2.5 | 1.0 | 3.1 | 1.0 | 1.9 |
| 4.0 | 6.0 | 4.0 | 8.0 | 4.0 | 4.1 |
| 8.0 | 12.7 | 8.0 | 17.4 | 8.0 | 8.1 |
| 12.0 | 19.5 | 12.0 | 26.9 | 12.0 | 12.1 |
| 16.0 | 28.3 | 16.0 | 39.1 | 16.0 | 17.4 |
| 20.0 | 37.1 | 20.0 | 50.8 | 20.0 | 23.4 |
| 24.0 | 45.1 | 24.0 | 59.7 | 24.0 | 30.5 |
| 28.0 | 55.7 | 28.0 | 68.2 | 28.0 | 43.2 |
| 32.0 | 66.7 | 32.0 | 76.5 | 32.0 | 56.9 |
| 36.0 | 73.9 | 36.0 | 81.2 | 36.0 | 66.5 |
| 40.0 | 80.2 | 40.0 | 85.1 | 40.0 | 75.2 |
| 44.0 | 83.6 | 44.0 | 87.2 | 44.0 | 80.0 |
| 48.0 | 85.3 | 48.0 | 88.2 | 48.0 | 82.4 |
| 52.0 | 86.8 | 52.0 | 89.1 | 52.0 | 84.5 |
| 56.0 | 87.3 | 56.0 | 89.4 | 56.0 | 85.2 |
| 60.0 | 89.7 | 60 | 91.2 | 60 | 88.3 |

The release profiles present an almost linear release for at least 2 months according to the present invention.

The effect of PLGA mixtures where the polymers were mixed in situ was also assessed. This can be done either by dissolving the two polymers in DCM before emulsification (one dispersed phase) or by emulsification of one dispersed phase containing one of the polymers and subsequently the emulsification of the second dispersed phase containing the second polymer in the same continuous phase (two different dispersed phases).

The dissolution profiles of these formulations are again similar to the profiles presented in table 6.

1. A pharmaceutical formulation, comprising:
microparticles,
wherein the microparticles comprise Tacrolimus, a first polymer, and a second polymer,
wherein the first and second polymer differ from each other,
wherein each of the first and second polymers is a poly(D,L-lactide-co-glycolide) polymer,
wherein each of the first and second polymers has an identical lactide to glycolide ratio, and
wherein each of the first and second polymers has a different molecular weight.

2. The formulation of claim 1, wherein each of the first and second polymers has a ratio of lactide to glycolide 50:50.

3. The formulation of claim 1, wherein the first and second polymers each have a different weight average molecular weight in a range of from 15,000 to 80,000 Da.

4. The formulation of claim 3, wherein the first and second polymers each have a different weight average molecular weight in a range of from 15,000 to 58,000 Da.

5. The formulation of claim 3, wherein the first and second polymers each have a different weight average molecular weight in a range of from 17,000 to 50,000 Da.

6. The formulation of claim 1, wherein the first polymer has a weight average molecular weight in a range of from 15,000 to 30,000 Da, and
wherein the second polymer has a weight average molecular weight in a range of from 30,000 to 80,000 Da.

7. The formulation of claim 6 wherein the first and second polymers have respective weight average molecular weights of 17,000 Da and 50,000 Da.

8. Microparticles, comprising:
Tacrolimus;
a first polymer; and
a second polymer,
wherein the first and second polymer differ from each other,
wherein each of the first and second polymers is a,
wherein each of the first and second polymers has an identical lactide to glycolide ratio, and
wherein each of the first and second polymers has a different molecular weight.

9. The formulation of claim 1, comprising two different microparticle types in a ratio of from 70:30 to 30:70.

10. The formulation of claim 1, wherein two different microparticles have a particle size as measured by laser light diffraction in a range of from 10 to 200 microns.

11. The formulation of claim 1, wherein the first and second polymers have a total concentration the microparticles in a rang of from 5 to 13% w/w.

12. The formulation of claim 1, which is configured to be reconstituted with a diluent before intramuscular or subcutaneous administration.

13. The formulation of claim 1, wherein the diluent comprises carboxymethylcellulose sodium, mannitol, sodium chloride, sodium hydroxide, polysorbate, acetic acid, sodium dihydrogen phosphate monohydrate, and/or disodium phosphate heptahydrate.

14. The formulation of claim 1, which is administered by intramuscular or subcutaneous injection.

15. The formulation of claim 1, which is configured to be administered once every two months.

16. The formulation of claim 1, wherein the Tacrolimus in the microparticles is at a loading in a range of from 20 to 30% w/w.

17. A process for preparing the microparticles of claim 8, the process comprising:
dissolving the first and second polymers under stirring in solvent, to obtain a polymer solution;
adding Tacrolimus in the polymer solution under stirring to form a dispersed oil phase;
preparing a continuous phase, comprising water for injection, poly(vinyl alcohol), and a buffering agent and keeping the continuous phase under a first controlled temperature;
mixing and emulsifying the dispersed and the continuous phases using a high shear rotor-stator continuous flow disperser or an overhead stirrer to form a suspension;
subjecting the suspension to solvent extraction and evaporation by stirring under a second controlled temperature and air flow for removal of organic solvents and microparticles solidification;
collecting the microparticles solidified onto sieves and washing the microparticles with water; and
drying the microparticles under vacuum.

18. A process for preparing the microparticles of claim 8, the process comprising:
(i) dissolving the first polymer under stirring in a solvent to obtain a first polymer solution;
adding Tacrolimus in the first polymer solution under stirring to form a first dispersed oil phase;
preparing a first continuous phase comprising water for injection, poly(vinyl alcohol), and a buffering agent and keeping the first continuous phase under a first controlled temperature;
mixing and emulsifying the first dispersed and the first continuous phases using a high shear rotor-stator continuous flow disperser or an overhead stirrer to form a first suspension;
(ii) dissolving the second polymer with a different molecular weight than the first polymer under stirring in dichloromethane, to obtain a second polymer solution;
adding Tacrolimus in the second polymer solution under stirring to form a second dispersed oil phase;
preparing a second continuous phase comprising water for injection, poly(vinyl alcohol), and a buffering agent, and keeping the second continuous phase under a second controlled temperature;
mixing and emulsifying the second dispersed and the second continuous phases using a high shear rotor-stator continuous flow disperser or an overhead stirrer to form a second suspension;
(iii) mixing the first and second suspensions containing the first polymer and the second polymer together to form a mixture and subjecting the mixture to solvent extraction and evaporation by stirring under a third controlled temperature and air flow for removal of organic solvents and microparticles solidification;
collecting the microparticles formed onto sieves and washing the microparticles with water; and
drying the microparticles under vacuum.

19. A process for preparing the microparticles of claim 8, the process comprising:
(i) dissolving the first polymer under stirring in a solvent to obtain a first polymer solution;
adding Tacrolimus in the first polymer solution under stirring to form a first dispersed oil phase;
preparing a first continuous phase comprising water for injection, poly(vinyl alcohol), and buffering agent and keeping the first continuous phase under a first controlled temperature;
mixing and emulsifying the first dispersed and the continuous phases using a high shear rotor-stator continuous flow disperser or an overhead stirrer to form a first suspension;
subjecting the suspension to solvent extraction and evaporation by stirring under a second controlled temperature and air flow for removal of organic solvents and first microparticles solidification;
collecting the first microparticles formed onto sieves and washing the first microparticles with water;
drying the first microparticles are dried under vacuum;
(ii) dissolving the second polymer with a different molecular weight to the first polymer under stirring in a solvent to obtain a second polymer solution;
adding Tacrolimus in the second polymer solution under stirring to form a second dispersed oil phase;
preparing a second continuous phase comprising water for injection, poly(vinyl alcohol), and a buffering agents and keeping the second continuous phase under a third controlled temperature;
mixing and emulsifying the second dispersed and the continuous phases using a high shear rotor-stator continuous flow disperser or an overhead stirrer to form a second suspension;
subjecting the second suspension to solvent extraction and evaporation by stirring under controlled temperature and air flow for removal of organic solvents and second microparticles solidification;
collecting the second microparticles formed onto sieves and washing the second microparticles with water;
drying the second microparticles under vacuum;
(iii) after drying the first and second microparticles, physically mixing the first and second microparticles.

20. The process of claim 17, wherein a ratio of the first polymer to the second polymer is in a range of from 70:30 to 30:70.

21. The process of claim 17, wherein the solvent for the dissolving is an organic solvent.

22. The process of claim 17, wherein the solvent comprises ethyl acetate, tetrahydrofuran, acetonitrile, dichloromethane, chloroform, and/or acetone.

23. The process of claim 17, wherein the solvent is dichloromethane.

24. The process of claim 17, wherein the first controlled temperature of the continuous phase is lower than 20° C.

25. The process of claim 17, wherein the first controlled temperature of the continuous phase is in a range of from 5 to 10° C.;

26. A process for prophylaxis of transplant rejection in an adult recipient for adult kidney, liver, or heart allograft, for treating and/or reducing organ rejection after transplantation, graft-versus-host diseases by medulla ossium transplantation, for treating and/or reducing an autoimmune disease, for treating and/or reducing an infectious disease, the process comprising:
providing to the adult in need thereof an effective amount of the pharmaceutical formulation of claim 1.

27. The formulation of claim 1, configured for intramuscular and/or subcutaneous administration with a dual chamber syringe or a kit comprising syringe pre-filled with a diluent and microparticles existing in a separate vial.
